(19) United States
(12) Patent Application Publication
(10) Pub. No.: US 2010/0036287 A1
(43) Pub. Date: Feb. 11, 2010

(54) Method and Device for Detecting Parameters for the Characterization of Motion Sequences at the Human or Animal Body

(76) Inventor: Michael Weber, Potsdam (DE)

Correspondence Address: CHERNOW KATZ, LLC, 721 DRESHER RD, SUITE 1100, HORSHAM, PA, 19044, US

(21) Appl. No.: 12/329,084

(22) Filed: Dec. 5, 2008

(30) Foreign Application Priority Data
Aug. 8, 2008 (DE) 102008037027.4
Oct. 21, 2008 (DE) 102008052506.9

(51) Int. Cl. A61B 5/11; G01N 3/20; G01N 3/08; G01V 7/00

(52) U.S. Cl. 600/595; 73/849; 73/826; 73/382 R

(57) ABSTRACT

The invention relates to a bending sensor for detecting function parameters for the characterization of motion sequences at the human or animal body. The bending sensor comprises a fixing element 20, in particular a fixing plaster, for fixing the bending sensor on the skin of the human or animal body. Furthermore, a bending-sensitive detector 10 for detecting bending parameters of the bending sensor is provided. The detected bending parameters such as, for instance, the bending angle, the bending rate, and/or the bending acceleration are stored in a data memory 30. The fixing element is extensible and comprises an extensible cavity for accommodating a measuring sensor of the detector. The measuring sensor is fixed at a reference point of the fixing element in the cavity.

### CROSS-REFERENCE TO RELATED PATENT APPLICATIONS

The present application claims priority under 35 U.S.C. §119(a) to German Application No. 102008037027.4, filed Aug. 8, 2008, and German Application No. 102008052406.9, filed Oct. 21, 2008, the entireties of which are hereby incorporated by reference.

### FIELD OF THE INVENTION

The present invention relates to a method for detecting function parameters for the characterization of motion sequences at the human or animal body, and to a bending sensor for performing the method.

### BACKGROUND OF THE INVENTION

Backache is a more or less strong ache of the human back which may have very different reasons. Physicians speak of dorsalgia or lumbalgy if the loin-sacrum region is concerned. The most frequent reason for a dorsalgia probably consists in a dysfunction of the joints in the region of the spine. However, approx. 90 per cent of any chronical (recurrent or persistent) backache is still unspecific—i.e. in the scope of medical examinations no findings can ultimately be made which explain the disorders sufficiently. At present, it is not possible to objectively detect movement functions, for instance, for treating unspecific backache. Merely structural findings by means of imaging diagnostic methods are usual (which are, however, not relevant in the case of unspecific backache).

### SUMMARY OF THE INVENTION

It is therefore an object of the present invention to provide a device that enables the detection of function parameters for the characterization of motion sequences at the human or animal body, so that unspecific or chronic disorders may be assigned to the motion patterns detected. The detection of the bending information is to take place predominantly in the region of the lumbar spine since the reason for a majority of any backache is to be seen here.

The measurement of dynamic function parameters is an unexplored, but very important field in spine medicine. The bending sensor according to the invention provides the possibility of getting an insight in everyday movements, in the function, the mobility, and the activity rate. The detection of motion parameters by means of the bending sensor according to the invention for orthopedy and spine medicine may be considered to be an analogon to long-term ECG.

The object is solved by the bending sensor for detecting function parameters for the characterization of motion sequences at the human and animal body in accordance with the enclosed claim 1. The bending sensor comprises a fixing element, in particular a fixing plaster, for fixing the bending sensor on the skin of the human or animal body. Furthermore, a bending-sensitive detector is provided for detecting bending parameters of the bending sensor. The bending parameters detected, such as the bending angle, the bending rate, and/or the bending acceleration, are stored in a data memory. The fixing element is extensible and comprises an extensible cavity for incorporating a measuring sensor of the detector. The measuring sensor is fixed at a reference point of the fixing element in the cavity.

A sensor or measuring sensor is a technical component that is adapted to quantitatively detect particular physical or chemical characteristics as a measurement. These measurements are converted to parameters (usually electric signals) that can be processed further.

The bending sensor according to the invention, for instance, is to detect the bending of the back. Bending designates the deformation of a body under a load that acts vertically on a body axis. For detecting the dynamics of motion sequences at the human body (for instance, at the back in the region of the lumbar spine), one or a plurality of measuring sensors are to be fixed on the skin by means of a fixing element, in particular a fixing plaster, in an appropriate position. The bending of the back leads to an extension of the skin. Extension is the relative dimension change (prolongation or contraction) of a body under strain. If the dimension of the body increases, one speaks of a positive extension, otherwise of a negative extension or compression. The extension of the skin on bending of the back amounts to approx. 50% of length change in the region of the lumbar spine in the case of a flexion movement. Therefore, the fixing element has to be flexible so that it does not loosen from the skin due to the length change discussed. The measuring sensors are fixed on the human skin (preferably at the left and at the right of the spine for detecting movements of the back) by means of flexible fixing elements. In so doing, the sensor band is connected with the fixing element via a reference point only and slides for the rest freely inside the cavity provided by the fixing element. The cavity quasi forms a guide channel in which the measuring sensor is indeed held at the body to follow the bending thereof, but without having to follow the length change of the skin.

The fixing element preferably has an elastic bottom layer with a biocompatible thermally activatable adhesive layer for application on the skin. Additionally, an elastic top layer is provided such that the cavity is formed between the top layer and the bottom layer. The cavity is thus no rigid body, but its design and extension depends on the integrated measuring sensor and the state of the elastic top and bottom layers. The top and bottom layers, however, see to it that the measuring sensor is held at a body, for instance, the back, such that it follows its bending without being affected by the corresponding length change of the skin.

A preferred embodiment of the bending sensor according to the invention comprises a measuring sensor that comprises an optical fiber. The optical fiber possesses a transmission function that changes as a function of a bending of the optical fiber. Optical fibers are lines for transmitting light in the visible and in the ultraviolet or infrared ranges. Examples of optical fibers are fiber optic cables, glass fibers, polymeric optical fibers, or other light-conductive components of plastics as well as fiber optic components. Every optical fiber possesses a transmission function. The transmission function of an optical fiber describes the relation between input and output signals for different frequencies of the light coupled in. The bending of the optical fiber results in the change of the transmission function, i.e. a bent optical fiber couples out less light in relation to the input signal than an unbent optical fiber. By the determination of the transmission function it is thus possible to detect the bending of the optical fiber.

The coupling out of light may, for instance, take place at the ends of the fibrous optical fiber. By means of the light coupled out it is to be detected whether and to what extent the optical fiber is bent; it is, however, not readily possible to detect a bending direction of the optical fiber. The optical fiber therefore preferably comprises an additional lateral aperture for the coupling out of light. The aperture is positioned on the surface of the optical fiber. As an aperture, a tapered depression or notch may in particular be provided. The lateral aperture constitutes a dispersion point that is further opened or closed on bending, so that the light output at the end of the fiber increases or decreases (linear signal for positive and negative bending direction). Furthermore, the lateral aperture determines where the bending measured occurs, i.e. the bending may be detected irrespective of the location. Preferably, a plurality of parallel optical fibers with lateral apertures provided at different segments are used, so that the bending may be detected at different locations.

In accordance with a preferred embodiment of the present invention, the measuring sensor comprises one or a plurality of strain gauges. Strain gauges are measuring means for detecting extending deformations. They change their impedance already with slight deformations and are used as extension sensors.

The electric impedance of the strain gauge changes with an extension or contraction of the strain gauge. By measuring the impedance, in particular the electric resistance, it is possible to determine the extent of the length change of the strain gauge. If the strain gauge is, as provided in accordance with the invention, accommodated in a cavity forming a guiding channel at the object to be examined, for instance, the back, a bending of the back will result in a corresponding bending of the strain gauge, so that the strain gauge changes its length.

The bending sensor according to the invention preferably comprises a detector comprising a detection device. The detection device detects a change of the electric impedance of the strain gauge or a change of the transmission function of the optical fiber. Thus, the bending sensor possesses a measure for the bending of the object to be examined.

The detector of the bending sensor according to the invention preferably comprises a substrate that guarantees tensile strength and that is elastically bendable on which the measuring sensor is fastened. The substrate is to take care that possibly occurring tensile stresses or compressive stresses do not cause any length change of the strain gauge. This is because such a length change would otherwise be interpreted wrongly as a measure for the bending of the object of examination. A suitable substrate that permits bending, but prohibits the stretching or compression of the strain gauge is spring steel. Spring steel is a steel that has a higher strength vis-a-vis other steels. A work piece of spring steel can be bent to a tension determined by the material (limit of elasticity) to then return elastically to the initial state without permanent deformation. The material property that enables this is elasticity. The substrate need not be spring band steel. It may also be FR 4. FR 4 is a designation for the fire resistance of a conductor board material; FR 4 is the common standard for consumer electronics.

In accordance with a preferred embodiment of the present invention, the detector comprises a plurality of measuring sensors that are fixed on opposite sides of the substrate. The reliability of the data detected is of utmost necessity for the biomechanical application. Both measuring sensors substantially detect the same bending of the substrate which follows the bending of the object of examination, e.g. the back. Consequently, the bending is represented by two measurements of the measuring sensors which are to be detected simultaneously. The actual bending may consequently be detected with higher exactness and reliability.

The bending sensor according to a preferred embodiment comprises a detector comprising a plurality of measuring sensors for detecting the bending parameters in respectively different measurement zones. The measuring sensors may be arranged in a cascaded or in an overlapping manner. Thus, it is possible to metrologically detect the bending information resulting from the body movement irrespective of the location. Every bending-sensitive measurement zone detects the respective curvature applied in its measuring section in a positive or negative bending direction in at least one spatial plane. The measuring sensors arranged in the different measurement zones are preferably triggered in a temporally offset manner, in particular with a clock frequency of 1 kHz or more, the more the better (pulse operation=current-saving). Thus, the detected data amount is substantially reduced since only data of one single measuring sensor are read out at each point in time. Simultaneously, the read-out frequency is large enough to detect the dynamics of the movement irrespective of the location. For instance, with a trigger frequency of 1 kHz and, for instance, 10 bending zones, a read-out frequency of 100 Hz becomes possible, which enables a dynamic detection of movement (rate, acceleration of bending).

Furthermore, the bending sensor according to the invention may comprise a position sensor for detecting the position of the measuring sensors relative to the gravitation field of the earth or to the earth's magnetic field. The gravitation field of the earth predetermines a constant direction. The measurement of the bending irrespective of the location enables to detect the bending in a particular plane relative to the direction of the gravitation field. Thus, it is, for instance, possible to determine how the back is bent for the lifting of loads.

The position sensor may also predetermine a starting vector for the detection of the bending information. The position sensor detects the position of the sensor at the back prior to the beginning of the bending measurement. The subsequently detected bending constitutes a relative deviation vis-a-vis the orientation of the starting vector.

The data detected by the detector are preferably output as digitized electronic signals by the detection device and are stored in an electronic data memory. As a data memory, a flash memory such as a SD memory card or a micro SD card may, for instance, be used. The flash memory offers the advantage that it is relatively small and light and preserves the stored data without the data memory having to be connected to a power supply. The flash memory may be fixed to the object of examination along with the fixing element. The flash memory, however, needs a current supply for the reading in or out of data. Alternatively, the data memory may also be provided in a separate memory unit. The data transmission will then be performed in a wire-bound or a wireless manner to the external data memory. This embodiment enables to design the electronic of the bending sensor distinctly smaller.

The fixing element according to the invention may in particular comprise a readable identification memory unit. The identification memory unit comprises an electronic identification for identifying the fixing element. It is thus possible to identify the fixing element so as to prevent that it is improperly exchanged for some other product. This feature in particular serves the quality assurance since the identification allows to ensure that only the bending sensor provided for measurement can be used with the fixing element.

As an identification memory unit for identifying the fixing element, a RFID transponder for the wireless reading out of the electronic identification may, for instance, be provided. The English term Radio Frequency Identification (RFID) designates a system for identification by means of electromagnetic waves. A RFID system consists of a transponder that is positioned at or in the object to be identified and that characterizes same, and of a reader for reading out the transponder identification. The detector according to the invention may then be provided with a reader for reading out the identification in the RFID transponder. The reading out is performed via an antenna, wherein data lines that are already available for measurement data transfer are used as an antenna, wherein they are modulated e.g. with 13.2 MHz and are thus made useful as an antenna, which makes further electric lines for an antenna dispensable. These measures ensure that neither the detector nor the fixing element can be exchanged for another product. A distinct allocation to the producer and to the number of applications is thus enabled. A multiple use of the fixing element is thus also excluded.

The measuring sensor(s) is/are preferably included by means of lamination, extrusion, casting, welding, or heating of a heat shrinkable tubing in a container that is closed against environmental influences. This feature is meant to ensure that the measuring sensor is protected from potentially harmful environmental influences such as moisture or pollution. In particular if the bending sensor according to the invention is used for long-time examinations over days or weeks is such a protection necessary.

The bending parameters detected are preferably used to determine a plurality of dynamic parameters. In particular the bending angle as a function of the time and/or the place, the bending rate as a function of the time and/or the place, the bending acceleration as a function of the time and/or the place, the Fourier transformation of the functions of the bending angle, the bending rate, and/or the bending acceleration can be derived.

The bending data detected are preferably entered in a histogram for the graphical representation of the frequency distribution of the dynamic parameters. A histogram is a graphic representation of the frequency distribution of measurements. One starts out from the data that are classified by size and subdivides the entire range of the sample in classes. Over every class an area is established whose size is proportional to the class-specific frequency. In particular the bending angle, the bending rate, or the bending acceleration are illustrated in such histograms. A frequency distribution of the position of the bending sensor may also be represented as a histogram or as a gray value in a coordinate system. The bending parameters detected are compared with average bending parameters so as to indicate aberrations in the movement parameters. Such a comparison is facilitated by the histogram representation.

Preferably, further parameters such as, for instance, pain parameters, posture parameters, mobility parameters are detected simultaneously with the bending parameters, and a statistic correlation is made between the bending parameters and the further parameters. The statistic correlation, however, does not express whether a causal coherence exists between the different parameters. The recognition of such coherences by a scientist or a physician is, however, facilitated if corresponding statistic correlations can be represented.

The detecting of bending parameters of the bending sensor is preferably performed over a period of at least 24 hours to enable a long-time analysis. The application is consequently performed in analogy to the long-time ECG—measurements are possible at arbitrary periods up to 24 hours. The detecting of bending parameters may in particular be performed in addition to treatment so as to detect a positive or a negative correlation between therapeutic measures and the movement parameters detected.

### BRIEF DESCRIPTION OF THE DRAWINGS

Preferred embodiments of the present invention will be described in the following with reference to the enclosed drawing.

FIG. 1 shows by way of example the application of a bending sensors according to the invention on the back of a person.

FIG. 2 shows a first embodiment for a detector of a bending sensor according to the invention.

FIG. 3 shows a Cartesian coordinate system in which the dependence of an electric change of resistance (ΔR) of the detector on a bending angle (Φ) is illustrated.

FIG. 4 shows a schematic view of a second embodiment for a bending sensor according to the invention.

FIG. 5 shows a section view of the bending sensor of the second embodiment in a cutting plane 130.

FIG. 6 shows a third embodiment for a bending sensor according to the invention.

FIG. 7 shows a section view of the bending sensor of the third embodiment in a cutting plane 130.

### DETAILED DESCRIPTION OF THE DRAWINGS

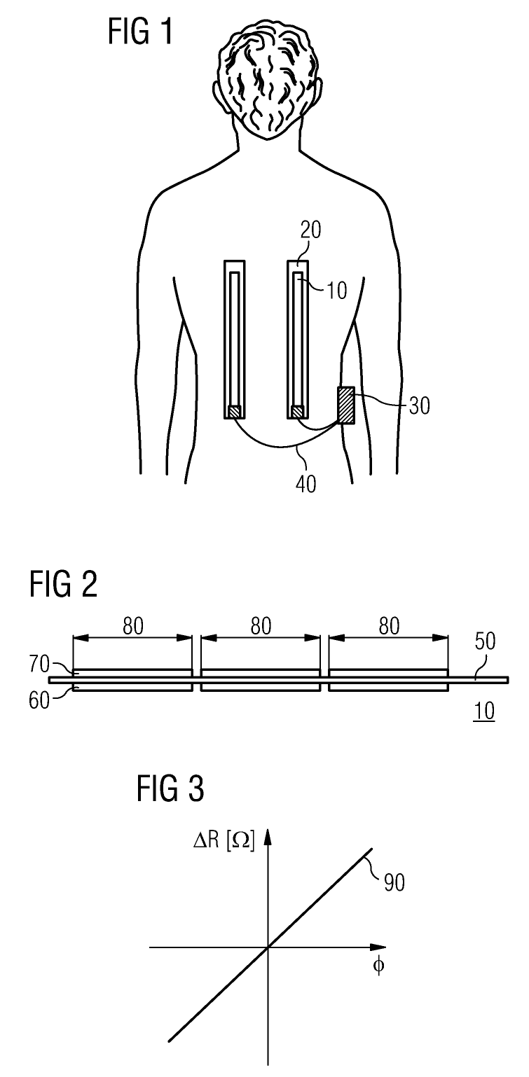

FIG. 1 exemplifies the application of a bending sensor according to the invention on the back of a person. A two-strip bending-sensitive detector 10 is shown, the strips of which are applied on the back of the person side by side. Strip-shaped elastic bands extend in FIG. 1 along the back parallel to the spine. They are fixed on the back by means of fixing elements 20. A data line 40 is provided which transmits the detected bending parameters to an electronic data memory 30.

FIG. 2 shows a lateral view of the strip-shaped detector section 10 of FIG. 1. The detector 10 comprises a strip-shaped substrate 50. On the opposite sides of the strip-shaped substrate 50, strain gauges 60 and 70 are arranged. The oppositely arranged strain gauges each define a measurement zone of the detector. The strain gauges 60 and 70 are measuring devices for the detection of extending deformations. They change their impedance with slight deformations already and are used as extension sensors. Once the person illustrated in FIG. 1 bends her or his back, the strain gauges 60 and 70 are each extended. This mechanical deformation changes the electric resistance of the strain gauges 60 and 70.

FIG. 3 shows the correlation between the mechanical deformation of the strain gauges and the change of the electric resistance in a Cartesian coordinate system. On the abscissa the bending angle Φ is illustrated. The bending angle Φ is a simple function of the extension of the strain gauge. Every single bending-sensitive measurement zone of a sensor band is calibrated, wherein the calibration curves are stored on the sensor band electronic. The ordinate of the coordinate system illustrates the change of the electric resistance ΔR vis-à-vis a resistance of an undeformed strain gauge. Reference number 90 represents the calibration curve; it indicates the functional relation between the change of resistance ΔR and the bending angle Φ. The calibration curve 90 in FIG. 3 is a simple straight line that runs through the origin of the coordinate system. This curve shape is illustrated as an example only. The calibration curves 90, however, deviate from the straight line in the case of larger bending angles Φ. The calibration is necessary to enable a detection of the bending angles which is as exact as possible. As sensor elements, optical fibers with a surface structuring may also be used in the region of the bending-sensitive measurement zones in addition to strain gauges. In addition to the Ohmic resistance, changes of the electric capacitance or the inductance may also be used as a measure for the bending angle. In general, the complex alternating current resistance Z or the impedance, respectively, may be plotted against the bending angle to be detected as a calibration curve.

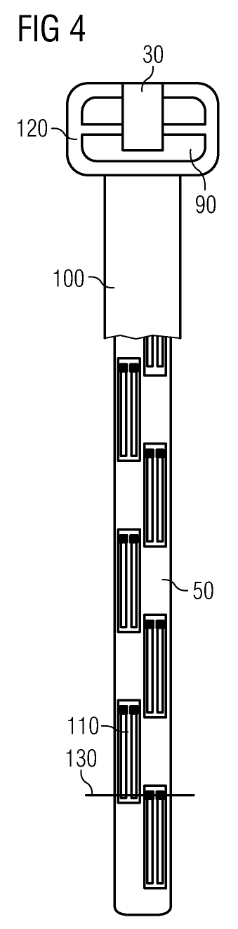

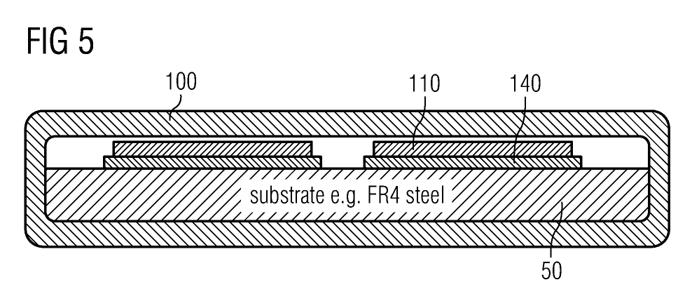

FIG. 4 shows a further embodiment of the bending sensor according to the invention. The bending sensor comprises a unit for the detector electronic 120 which is connected to a bending-sensitive detector 100. The bending-sensitive detector comprises a strip-shaped substrate 50. Along the strip-shaped substrate 50, a plurality of strip-shaped strain gauges 100 are arranged. The longitudinal axis of the strain gauges 110 is oriented parallel to the longitudinal axis of the strip-shaped substrate 50. Adjacent strain gauges 110 are each placed in sections side by side on the strip-shaped substrate, so that an initial section of a first strain gauge 110 is always positioned adjacent to an end section of a subsequent strain gauge 110. Reference number 130 designates a cutting plane through the bending-sensitive detector where the initial and end sections of two adjacent strain gauges are arranged side by side on the strip-shaped substrate.

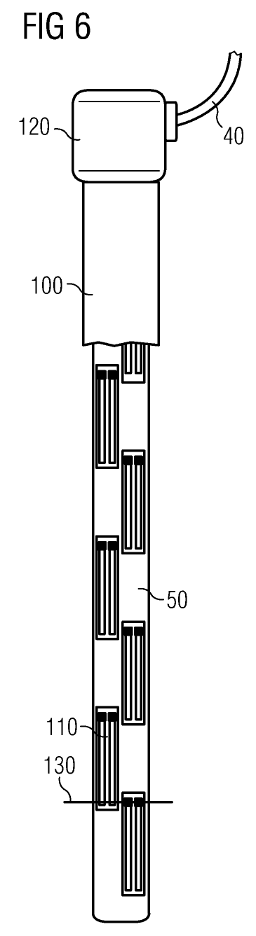

FIG. 5 shows a cross-section of the bending-sensitive detector 10 of FIG. 4 in the cutting plane 130. The substrate 50 illustrated in FIG. 5 is preferably manufactured of spring steel. This material has sufficient strength and flexibility to accommodate the strain gauges 110. FIG. 5 shows that the strain gauges 110 are fixed on the substrate 50 by means of an adhesive layer 140. As an adhesive layer 140, an epoxy resin may, for instance, be used which provides simultaneously an efficient electrical insulation of the strain gauge from the substrate. The substrate 50 and the strain gauges are enclosed by a casing 100. A heat shrinkable tubing is preferably used as a casing.

FIG. 6 shows a further embodiment of the present invention. Reference number 120 in FIG. 6 characterizes the detector electronic of the bending sensor illustrated. It is preferably connected via a data line with an external data memory that is not illustrated in FIG. 6. Thus, it is possible to reduce the dimension and the weight of the bending sensor as a whole. It is, of course, also possible to transmit the detected movement parameters via a wireless connection, for instance, Bluetooth (IEEE 802.15.1), WLAN (IEEE 802.11), or UMTS. The evaluation and storage of the detected movement parameters may thus be assigned to external computers. It is in particular possible to evaluate the detected data in real time so as to convey to the carrier of the bending sensor again information about the reduction or prevention of backaches.

The bending sensor illustrated in FIG. 6 further comprises a substrate 50 on which strain gauges 110 are arranged in a cascaded manner. The term cascading here means alone the spatial arrangement of the strain gauges. The strain gauges 110 are not connected in series, but are connected parallel. Thus, it is possible to trigger and read every strain gauge individually. Every strain gauge 50 is intended to detect movement parameters at different locations or measurement zones, respectively, so that a representation of movement parameters irrespective of the location may be obtained. The strain gauges 110 are each consecutively arranged along a longitudinal axis of the substrate 50. FIG. 6 merely shows a top view of the bending sensor. On the rear of the bending sensor which is not illustrated, strain gauges are again arranged in the same manner as on the front that is illustrated.

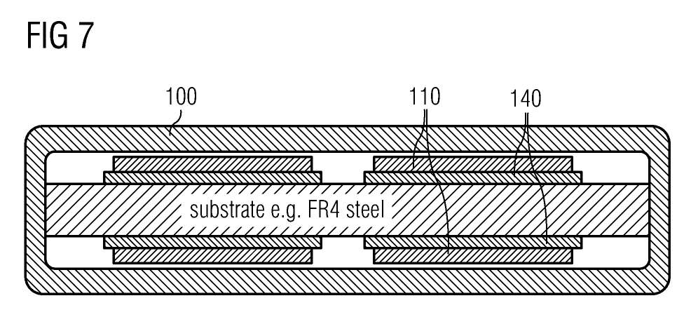

FIG. 7 shows a cross-section of the bending sensor of FIG. 6 along a cutting plane 130. At the opposite sides of the substrate 50, strain gauges 110 are arranged. By means of an adhesive layer 140 the strain gauges 110 are each fixed on the substrate 50. By combining two parallel (sandwich structure) measuring sensors that are oriented in an inversive or an aligned manner per bending-sensitive measurement zone, the measuring accuracy, the measurement range, the immunity to disturbances, and thus the reproducibility of the bending signals is increased since thermal, mechanical, and electromagnetic influences as well as degradation of the measuring sensors are compensated for.

### LIST OF REFERENCE SIGNS

10 bending-sensitive detector
20 fixing element
30 data memory
40 data line
50 substrate
60 upper strain gauge
70 lower strain gauge
80 measurement zones
85 calibration curve
90 current supply
100 casing
110 strain gauge
120 detector electronic
130 cutting plane
140 adhesive for strain gauges

While the invention has been described and illustrated in sufficient detail that those skilled in this art can readily make and use it, various alternatives, modifications, and improvements should become readily apparent without departing from the spirit and scope of the invention.

What is claimed is:

1. A bending sensor for detecting function parameters for the characterization of motion sequences at the human or animal body, comprising:
a fixing element (20), in particular a fixing plaster, for fixing the bending sensor on the skin of the human or animal body;
a bending-sensitive detector (10) for detecting bending parameters of the bending sensor, in particular a bending angle, a bending rate, and a bending acceleration, and
a data memory (30) for storing the detected bending parameters; and
characterized in that the fixing element is extensible and comprises an extensible cavity for accommodating a measuring sensor of the detector, wherein the measuring sensor is fixed at a reference point of the fixing element in the cavity.

2. The bending sensor according to claim 1, wherein the fixing element (20) comprises an elastic bottom layer with a biocompatible thermally activatable adhesive layer for application on the skin, and an elastic top layer, wherein the cavity is formed between the top layer and the bottom layer.

3. The bending sensor according to claims 1 or 2, wherein the measuring sensor comprises an optical fiber with a transmission function that changes as a function of a bending of the optical fiber.

4. The bending sensor according to claim 3, wherein the measuring sensor comprises a strain gauge with an electric impedance that changes with an extension or compression of the strain gauge.

5. The bending sensor according to claim 4, wherein the detector comprises a detection device, wherein the detection device detects a change of the electric impedance of the strain gauge (60, 70) or a change of the transmission function of the optical fiber.

6. The bending sensor according to claim 5, wherein the detection device is adapted to detect the change of the electric impedance of a strain gauge (60, 70) or the change of the transmission function of the optical fiber with a predetermined scanning frequency of, for instance, 100 Hz.

7. The bending sensor according to claim 6, comprising an electronic data memory for storing the digitized electronic signals output by the detection device.

8. The bending sensor according to any of claim 1, wherein the detector comprises a substrate that guarantees tensile strength and is elastically bendable on which the measuring sensor is fixed.

9. The bending sensor according to claim 8, wherein the substrate is manufactured of spring steel.

10. The bending sensor according to claims 8, wherein the detector comprises a plurality of measuring sensors that are fixed on opposite sides of the substrate.

11. The bending sensor according to any of the preceding claims 1, wherein the detector (10) comprises a plurality of measuring sensors for detecting the bending parameters in respectively different measurement zones (80).

12. The bending sensor according to claim 11, wherein the measuring sensors are arranged in a cascaded or overlapping manner.

13. The bending sensor according to any of claims 12, wherein the detection device is adapted to trigger the measuring sensors arranged in the different measurement zones (80) in a temporally displaced manner, in particular with a trigger frequency of at least 1 kHz.

14. The bending sensor according to any of claims 13, comprising a position sensor for detecting the position of the measuring sensors relative to the gravitation field of the earth or to the earth's magnetic field.

15. The bending sensor according to any of claims 14, wherein the fixing element (20) comprises a layer of spring steel for mechanical stabilization and electromagnetic shielding.

16. The bending sensor according to any of claims 15, wherein the fixing element (20) comprises a readable identification memory unit, wherein an electronic identification for identifying the fixing element is stored in the identification memory unit.

17. The bending sensor according to claim 16, wherein the identification memory unit comprises a RFID transponder for the wireless reading of the electronic identification.

18. The bending sensor according to claims 16, wherein the detector comprises a reader for reading the identification.

19. The bending sensor according to claim 17, wherein the RFID transponder uses as an antenna a data line that is already available for the transfer of measurement data.

20. A method for detecting function parameters for the characterization of motion sequences at the human or animal body, comprising the steps of:
fixing a bending sensor on a human or animal body;
detecting bending parameters of the bending sensor, in particular a bending angle, a bending rate, and a bending acceleration;
determining the body movement by means of the detected bending parameters;
providing an extensible fixing element for fixing the bending sensor, wherein the fixing element comprises an extensible cavity for accommodating a measuring sensor of the detector; and
fixing the measuring sensor at a reference point of the fixing element in the cavity.

21. The method according to claim 20, further comprising:
detecting a position of the bending sensor by means of a position sensor, wherein the position is detected relative to the gravitation field of the earth or to the earth's magnetic field.

22. The method according to claim 21 wherein the bending parameters are detected irrespective of the location in that a plurality of bending-sensitive measurement zones (80) of the bending sensor are used for the detection of bending parameters.

23. The method according to claim 22 wherein the bending parameters are detected irrespective of the time in that the bending parameters are detected with a predetermined scanning frequency of, for instance, 100 Hz.

24. The method according to claim 23, wherein the detected bending parameters are used to determine a plurality of dynamic parameters, in particular the bending angle as a function of the time and/or the place, the bending rate as a function of the time and/or the place, the bending acceleration as a function of the time and/or the place, the Fourier transformation of the functions of the bending angle, the bending rate and/or the bending acceleration.

25. The method according to claim 24, wherein a histogram is used for the graphic representation of the frequency distribution of the dynamic parameters, in particular the bending angle, the bending rate, or the bending acceleration.

26. The method according to claim 24, wherein a frequency distribution of the position of the bending sensor is represented as a histogram or as a gray value in a coordinate system.

27. The method according to claim 20, wherein the detected bending parameters are compared with average bending parameters so as to indicate aberrations in the movement parameters.

28. The method according to claim 20, wherein further parameters such as, for instance, pain parameters, posture parameters, mobility parameters, are detected simultaneously with the bending parameters, and a statistic correlation between the bending parameters and the further parameters is established.

29. The method according to claim 20, wherein the detection of bending parameters of the bending sensor is performed for a period of at least 24 hours so as to enable a long-time analysis.

30. The method according to claims 20, wherein the detection of bending parameters is performed in addition to treatment so as to detect a positive or a negative correlation between therapeutic measures and the movement parameters detected.
